(19) United States
(12) Patent Application Publication
(10) Pub. No.: US 2003/0096417 A1
(43) Pub. Date: May 22, 2003

(54) Vaccination against feline immunodeficiency virus

(76) Inventor: Laurent Bernard Fischer, Sainte Foy Les Lyon (FR)

Correspondence Address: FROMMER LAWRENCE & HAUG, 745 FIFTH AVENUE- 10TH FL., NEW YORK, NY, 10151, US

(21) Appl. No.: 10/157,319

(22) Filed: May 29, 2002

Related U.S. Application Data
(60) Provisional application No. 60/295,371, filed on Jun. 1, 2001.

(51) Int. Cl. C12Q 1/70; G01N 33/53; A61K 31/70; A01N 43/04; A61K 39/275; A61K 39/285

(52) U.S. Cl. 435/975; 514/44; 424/232.1; 435/5; 435/7.1

(57) ABSTRACT

The invention relates to a method for immunizing a Felidae against FIV, comprising:

first administering to the Felidae a first immunogenic composition comprising, in a pharmaceutically acceptable vehicle or excipient, a plasmid containing and expressing, in vivo, a polynucleotide encoding an FIV protein chosen among the group consisting of env, gag and gag/pro,

then administering to the same Felidae a second immunogenic composition comprising, in a pharmaceutically acceptable vehicle or excipient, a viral vector containing and expressing in vivo, a polynucleotide encoding an FIV protein chosen among the group consisting of env, gag and gag/pro, with the condition according to which a same FIV protein chosen among the group consisting of env, gag and gag/pro, is encoded by both the plasmid and the viral vector.

### RELATED APPLICATIONS

This application claims priority from U.S. Provisional application Serial No 60/295,371, filed Jun. 1, 2001.

### FIELD OF THE INVENTION

The present invention relates to immunization and vaccination against feline immunodeficiency virus. It also relates to vaccination kits.

Each document cited in this text (*application cited documents*) and each document cited or referenced in each of the application cited documents, is hereby incorporated herein by reference; and, technology in each of the documents incorporated herein by reference can be used in the practice of this invention.

### BACKGROUND OF THE INVENTION

Feline immunodeficiency virus (FIV) is a single-stranded RNA retrovirus of positive polarity belonging to the lentivirus family.

FIV, which is widely distributed throughout the world, essentially affects felines, in particular cats.

The disease is characterized by deterioration and suppression of the immune system, allowing the development of opportunistic diseases and possibly leading to death.

The FIV RNA molecule is composed of several open reading frames (ORFs) encoding structural proteins, in particular env and gag, viral enzymes, in particular pol and pro, and transactivators, in particular tat and rev. The rev ORF is composed of two exons.

Inactivated vaccines have been proposed, but, in order to be protective, require very large amounts of antigens, which poses problems for industrialization.

Other vaccinal approaches have been attempted, in particular the use of the envelope protein, in subunit form or expressed by a recombined expression vector. These studies have not produced satisfactory results. They have, moreover, demonstrated enhancement phenomena, resulting in a viraemia which occurs earlier in vaccinated animals than in control animals.

The article by Cuisiner (Cuisinier A. M. et al., Vaccine, 1997, 15(10), 1085-1094) recalls that, subsequent to vaccination attempts using inactivated viruses, it appeared that the gp120 surface glycoprotein would be determinant for protection. It recalls, however, that several vaccination attempts using recombined env proteins have not made it possible to induce protection. The authors describe experiments in which cats are vaccinated by intramuscular administration of DNA encoding FIV gp120 and p10 (nucleocapsid). Administering DNA encoding FIV gp120 induces only partial protection. When a combination of gp120 and p10 is used, no protection is observed.

On the other hand, the article by Richardson (Richardson J. et al., J. Virol., 1997, 71(12), 9640-9649) reports, with administration of plasmids encoding FIV env, an enhancement phenomenon. This phenomenon has also been observed in vaccination using recombined FIV proteins (Lutz H. et al., AIDS Research and Human Retroviruses, 1996, 12(5), 431-433) and vaccinia virus expressing FIV env (Osterhaus A. D. M. E. et al., AIDS Research and Human Retroviruses, 1996, 12(5), 437-441).

WO-A-98/21354 describes a protocol for vaccination against FIV, in which a recombined canarypox vector having FIV env and gag/pro as the insert is first administered, followed by inactivated FIV produced on T lymphocytes.

In Cuisinier A. M. et al., Vaccine, 1999, 17, 415-425, the administration of DNA expressing gp120 is here again the cause of enhancement phenomena.

Various vaccination approaches have therefore been studied, namely inactivated vaccines, subunit vaccines and recombined vaccines (viral vectors and DNA). To date, there is no satisfactory solution nor any clear direction for research.

### DESCRIPTION OF THE INVENTION

After considerable research efforts, the applicant has been able to develop a technique for vaccinating felines against FIV using recombined vaccines of the “viral vector and DNA (plasmid)” type. Notably, the technique developed makes it possible to use the expression of env without generating an enhancement problem.

Thus, a first subject of the invention is a method for immunizing and vaccinating felines against FIV, using an administration protocol comprising at least one primary administration and at least one booster administration using at least one common immunogen. The immunogenic preparations and the vaccines used in primary administration are different in nature from those used as a booster. This administration protocol is called “prime-boost”. The prime-boost protocol according to the invention comprises a primary administration with an immunogenic preparation or a vaccine comprising, in a pharmaceutically acceptable vehicle or excipient, a plasmid containing and expressing, in vivo, a polynucleotide encoding FIV env and/or gag and/or gag/pro, followed by a booster with an immunogenic preparation or a recombined vaccine comprising, in a pharmaceutically acceptable vehicle or excipient, a viral vector containing and expressing, in vivo, a polynucleotide encoding FIV env and/or gag and/or gag/pro, with the condition according to which at least one of the env or gag or gag/pro proteins is encoded by both the plasmids and the viral vectors.

The primary administration may comprise one or more administrations of the same plasmid-based immunogenic preparations or vaccines. Similarly, the booster administration may comprise one or more administrations of the same viral vector-based immunogenic preparations or vaccines. According to a particular embodiment of the invention, the protocol comprises two successive administrations of the same plasmid-based immunogenic preparation or vaccine, and then one administration of a viral vector-based immunogenic preparation or vaccine, as a booster.

The various administrations are preferably carried out 3 to 6 weeks apart, and more particularly about 4 weeks apart. According to a preferred mode, an annual booster, preferably using the viral vector-based immunogenic preparation or vaccine, is also envisaged. The animals are preferably at least 6 to 8 weeks old at the time of the first administration.

The plasmids and viral vectors used in the prime-boost protocol preferably express both env and gag or env and gag/pro.

For the purposes of the invention, the term “protein” encompasses the fragments, including peptides and polypeptides, having substantially the same immunological activity as the whole protein. The expressions env gene, gag gene, gag/pro gene, etc., thus include polynucleotide fragments encoding such protein fragments, peptides and polypeptides.

The polynucleotides, in order to control their expression, are functionally associated with a promoter and, optionally, with enhancers, with stabilizing sequences and/or with signal sequences for secretion of the protein.

Other FIV proteins may be expressed jointly with env and/or gag and/or gag/pro, in primary administration and/or as a booster. The tat protein and/or rev protein may in particular be expressed, preferably tat and optionally rev. They may be expressed in the same plasmids and/or viral vectors as those used for env and/or gag and/or gag/pro, or in separate plasmids and/or viral vectors.

According to a particular mode, several FIV strains are represented in the preparations or vaccines of the invention, i.e. the protocol comprises the administration of plasmids and of viral vectors comprising and expressing, in vivo, polynucleotides encoding env and/or gag and/or gag/pro from two or three or more FIV strains. Thus, polynucleotides from several FIV strains may be carried by a single viral vector or by separate viral vectors. For the plasmids, preference is given to separate plasmids, but a single plasmid carrying the polynucleotides from several FIV strains is not, however, excluded.

According to yet another mode, and as will be seen in greater detail later, the protocol may comprise the concomitant administration of one or more cytokines, either in protein form or by incorporating a polynucleotide encoding a cytokine into plasmids and/or viral vectors, those used to express the FIV proteins and/or others.

For producing the expression vectors according to the invention, various FIV strains, the organization of their genome and the nucleotide sequence of their genome are available to those skilled in the art. Useful FIV strains, such as the Petaluma strain are cited in the part Examples. Supplemental information on these strains and others, and their nucleotide sequences, is given at the beginning of the part Examples.

According to the invention, the primary administration comprises the use of plasmids which express the FIV protein(s) in vivo. The term “plasmid” refers to a DNA transcription unit comprising a polynucleotide according to the invention and the elements required for its expression in vivo. Preference is given to the circular plasmid form, which may or may not be supercoiled. The linear form also falls within the context of this invention.

Each plasmid comprises a promoter capable of ensuring, in host cells, the expression of the polynucleotide inserted under its control. It is, in general, a strong eukaryotic promoter. The cytomegalovirus immediate early (CMV-IE) promoter, of human or murine origin, or optionally of any other origin such as rat or guinea pig, is the preferred strong eukaryotic promoter. The CMV-IE promoter may comprise the actual promoter component, which may or may not be associated with the enhancer component. Reference may be made to EP-A-260 148, EP-A-323 597, U.S. Pat. No. 5,168,062, U.S. Pat. No. 5,385,839, U.S. Pat. No. 4,968,615 and WO-A-87/03905. Human (Boshart M. et al., Cell., 1985, 41, 521-530) or murine CMV-IE is preferred.

More generally, the promoter is either of viral origin or of cellular origin. As a strong viral promoter other than CMV-IE, mention may be made of the early promoter or the late promoter of the SV40 virus or the LTR promoter of the Rous Sarcoma virus. As a strong cellular promoter, mention may be made of the promoter of a cytoskeleton gene, such as, for example, the desmin promoter (Kwissa M. et al., Vaccine, 2000, 18(22), 2337-2344) or the actin promoter (Miyazaki J. et al., Gene, 1989, 79(2), 269-277).

By equivalence, the subfragments of these promoters, which conserve suitable promoter activity, are included in the present invention: e.g. the truncated CMV-IE promoters according to WO-A-98/00166. The notion of promoter according to the invention therefore includes the derivatives and subfragments which conserve suitable promoter activity, preferably substantially similar to that of the actual promoter from which they are derived. For CMV-IE, this notion comprises the actual promoter component and/or the enhancer component and the derivatives and subfragments.

The plasmids preferably comprise other elements for controlling expression. In particular, it is advantageous to incorporate stabilizing sequences of the intron type, preferably intron II of the rabbit β-globin gene (van Ooyen et al. Science, 1979, 206: 337-344).

As a polyadenylation (polyA) signal for the plasmids, use may in particular be made of that of the bovine growth hormone (bGH) gene (U.S. Pat. No. 5,122,458), that of the rabbit β-globin gene or that of the SV40 virus.

According to the invention, the booster administration comprises the use of viral vectors which express the FIV protein(s) in vivo. These viral expression vectors are advantageously avipox viruses (in particular canarypox, fowlpox) or attenuated mutants of the vaccinia virus.

For the poxviruses, those skilled in the art may refer to WO-A-90/12882, and more particularly, for the vaccinia virus, to U.S. Pat. No. 4,769,330; U.S. Pat. No. 4,722,848; U.S. Pat. No. 4,603,112; U.S. Pat. No. 5,110,587; U.S. Pat. No. 5,494,807; U.S. Pat. No. 5,762,938; for fowlpox, to U.S. Pat. No. 5,174,993; U.S. Pat. No. 5,505,941; U.S. Pat. No. 5,766,599; and for canarypox, to U.S. Pat. No. 5,756,103. As attenuated mutant of vaccinia virus, one may mention the MVA (Ankara strain) (Stickl H. and Hochstein-Mintzel V., Munch, Med. Wschr., 1971, 113, 1149-1153; Sutter G. et al., Proc. Natl. Acad. Sci. USA., 1992, 89, 10847-10851; commercial strain ATCC VR-1508; MVA is obtained after 570 passages of the Ankara vaccinia strain on chicken embryo fibroblasts), or the NYVAC (its construction is described in U.S. Pat. No. 5,494,807, in particular in examples 1 to 6; this patent also describes insertion of heterologous genes within insertion sites in this recombinant, and the use of appropriate promoters; see also WO-A-96/40241).

According to one of the preferred embodiments of the invention, the poxvirus expression vector is a canarypox virus, optionally attenuated, e.g. an ALVAC or a canarypox virus (for example of the Rentschler strain) which has been attenuated, in particular by more than 200 passages on chick embryo fibroblast (CEF) cells. An ALVAC strain canarypox virus was registered, on Nov. 14, 1996, with the American Type Culture Collection (ATCC) under the accession number VR-2547. A canarypox is commercially available at the ATCC under reference VR-111. Attenuated canarypox viruses are described in U.S. Pat. No. 5,756,103 and WO-A-01/05934.

Other attenuated poxviruses may be used, in particular attenuated fowlpoxes (e.g. TROVAC). Regarding the TROVAC poxvirus, those skilled in the art may refer to patent WO-A-96/40241. A number of fowlpox vaccinal strains are available, e.g. the vaccine DIFTOSEC CT® sold by Merial and the vaccine NOBILIS® sold by Intervet.

When the expression vector is an attenuated mutant of a vaccinia virus, the insertion sites for the polynucleotide(s) to be expressed are, in particular, the thymidine kinase (TK) gene, the haemagglutinin (HA) gene and/or the A-type inclusion body (ATI) region. Insertion of genes in the MVA virus is also described in several publications, e.g. in M. W. Carroll et al., Vaccine 1997, 15(4), 387-394; K. J. Stittelaar et al., J. Virol. 2000, 74(9), 4236-4243; G. Sutter et al., Vaccine 1994, 12(11), 1032-1040, to which the one skilled in the art may refer. The complete genome of MVA is described in G. Antoine, Virology 1998, 244, 365-396, which allows one to find other insertion sites and other promoters.

When it is a canarypox, the insertion sites are in particular located in, or consist of, ORFs C3, C5 and C6. When it is a fowlpox, the insertion sites are in particular located in, or consist of, ORFs F7 and F8.

Preferably, when the expression vector is a poxvirus, the polynucleotide to be expressed is inserted under the control of a poxvirus-specific promoter, in particular the vaccinia 7.5 kDa promoter (Cochran et al., J. Virology, 1985, 54, 30-35), the vaccinia 13L promoter (Riviere et al., J. Virology, 1992, 66, 3424-3434), the vaccinia HA promoter (Shida, Virology, 1986, 150, 451-457), the cowpox ATI promoter (Funahashi et al., J. Gen. Virol., 1988, 69, 35-47) or the vaccinia H6 promoter (Taylor J. et al., Vaccine, 1988, 6, 504-508; Guo P. et al., J. Virol., 1989, 63, 4189-4198; Perkus M. et al., J. Virol., 1989, 63, 3829-3836).

A subject of the invention is also the use, firstly, of plasmids containing and expressing, in vivo, the polynucleotide(s) encoding FIV env and/or gag and/or gag/pro, for producing a first immunogenic preparation or a first vaccine comprising the plasmids and a pharmaceutically acceptable vehicle or excipient, intended to be administered to felines as a primary administration and, secondly, of viral vectors containing and expressing, in viva, the polynucleotide(s) encoding FIV env and/or gag and/or gag/pro, for producing a second immunogenic preparation or a second vaccine comprising the viral vectors and a pharmaceutically acceptable vehicle or excipient, intended to be administered to the same felines as a booster, with the condition according to which at least one of the env or gag or gag/pro proteins is encoded by both the plasmids and the viral vectors, for vaccinating felines against FIV.

A subject of the invention is also the use of plasmids containing and expressing, in vivo, one or more polynucleotide(s) encoding FIV env and/or gag and/or gag/pro, for producing a vaccine comprising the plasmids and a pharmaceutically acceptable vehicle or excipient, intended, for vaccinating felines against FIV, to be administered to felines as a primary administration, the booster being effected using a vaccine comprising viral vectors containing and expressing, in vivo, one or more polynucleotide(s) encoding FIV env and/or gag and/or gag/pro and a pharmaceutically acceptable vehicle or excipient, with the condition according to which at least one of the env or gag or gag/pro proteins is encoded by both the plasmids and the viral vectors.

A subject of the invention is also the use of viral vectors containing and expressing, in vivo, one or more polynucleotide(s) encoding FIV env and/or gag and/or gag/pro, for producing a vaccine comprising the viral vectors and a pharmaceutically acceptable vehicle or excipient, intended, for vaccinating felines against FIV, to be administered to felines as a booster for a vaccine comprising plasmids containing and expressing, in vivo, one or more polynucleotide(s) encoding FIV env and/or gag and/or gag/pro and a pharmaceutically acceptable vehicle or excipient, with the condition according to which at least one of the env or gag or gag/pro proteins is encoded by both the plasmids and the viral vectors.

A subject of the present invention is also a kit for immunizing or for vaccinating Felidae against FIV, in particular intended to be used according to the administration protocol according to the invention, comprising, packaged separately:

an immunogenic preparation or a vaccine comprising, in a pharmaceutically acceptable vehicle or excipient, a plasmid containing and expressing, in vivo, a polynucleotide encoding FIV env and/or gag and/or gag/pro,

an immunogenic preparation or a recombined vaccine comprising, in a pharmaceutically acceptable vehicle or excipient, a viral vector containing and expressing, in vivo, a polynucleotide encoding FIV env and/or gag and/or gag/pro,

with the condition according to which at least one of the env or gag or gag/pro proteins is encoded by both the plasmids and the viral vectors.

The plasmids and viral vectors preferably express env and gag or env and gag/pro.

It goes without saying that all the characteristics described in the present application, which relate, for example, to the composition of the plasmids and viral vectors, the composition of the preparations and vaccines, the FIV polynucleotide combinations and the administration protocol, apply, under the same conditions, to the various subjects of the invention.

The notion of immunogenic composition covers any composition capable, once administered to the target species under the conditions of the invention, of inducing an immune response directed against FIV. The term “vaccine” is intended to mean a composition capable of inducing effective protection. The target species are the Felidae, preferably cats.

The pharmaceutically acceptable vehicles or excipients are completely known to those skilled in the art. By way of example, it may be a 0.9% NaCl saline solution or a phosphate buffer. The pharmaceutically acceptable vehicles or excipients also encompass any compound or combination of compounds which facilitate administration of the vector, in particular transfection, and/or which improve conservation.

The immunogenic compositions and the vaccines according to the invention preferably comprise one or more adjuvants, in particular selected from the usual adjuvants. The following are particularly suitable in the context of the present invention: (1) polymers of acrylic or methacrylic acid, polymers of maleic anhydride and of an alkenyl derivative, (2) immunostimulatory sequences (ISS), in particular oligodeoxyribonucleotide sequences having one or more non-methylated CpG motifs (Klinman D. M. et al., Proc. Natl. Acad. Sci. USA, 1996, 93, 2879-2883; WO-A1-98/16247), (3) an oil-in-water emulsion, in particular the SPT emulsion described on page 147 of “Vaccine Design, The Subunit and Adjuvant Approach” edited by M. Powell, M. Newman, Plenum Press 1995, and the MF59 emulsion described on page 183 of the same work, (4) cationic lipids containing a quaternary ammonium salt, (5) cytokines, or (6) combinations or mixtures thereof.

The oil-in-water emulsion (3), which is particularly suitable for the viral vectors, may in particular be based:

on light liquid paraffin oil (European Pharmacopoeia type);

on isoprenoid oil, such as squalane or squalene;

on oil resulting from the oligomerization of alkenes, in particular of isobutene or of decene;

of esters of acids or alcohols containing a linear alkyl group;

more particularly plant oils, ethyl oleate, propylene glycol di(caprylate/caprate), glyceryl tri(caprylate/caprate), propylene glycol dioleate;

esters of branched fatty alcohols or acids, in particular esters of isostearic acid.

The oil is used in combination with emulsifiers to form the emulsion. The emulsifiers are preferably nonionic surfactants, in particular:

esters, firstly, of sorbitan, of mannide (e.g. anhydromannitol oleate), of glycerol, of polyglycerol or of propylene glycol and, secondly, of oleic, isostearic, ricinoleic or hydroxystearic acid, these esters optionally being ethoxylated,

polyoxypropylene-polyoxyethylene block copolymers, in particular Pluronic®, especially L121.

Among the adjuvant polymers of type (1), preference is given to polymers of acrylic or methacrylic acid which are crosslinked, in particular crosslinked with polyalkenyl ethers of sugars or of polyalcohols. These compounds are known as carbomers (Pharmeuropa Vol. 8, No. 2, June 1996). Those skilled in the art may also refer to U.S. Pat. No. 2,909,462, which describes such acrylic polymers crosslinked with a polyhydroxylated compound having at least 3 hydroxyl groups, preferably no more than 8, the hydrogen atoms of at least three hydroxyls being replaced with unsaturated aliphatic radicals having at least 2 carbon atoms. The preferred radicals are those containing from 2 to 4 carbon atoms, e.g. vinyls, allyls and other ethylenically unsaturated groups. The unsaturated radicals may, themselves, contain other substituents, such as methyl. The products sold under the name Carbopol® (B F Goodrich, Ohio, USA) are particularly suitable. They are especially crosslinked with an allyl sucrose or with allyl pentaerythritol. Among these, mention may be made in particular of Carbopol® 974P, 934P and 971P.

The concentration of polymer of carbomer type in the final vaccinal composition may in particular range from 0.01% to 1.5% W/V, more particularly from 0.05% to 1% W/V, preferably from 0.1% to 0.4% W/V.

The cationic lipids (4) containing a quaternary ammonium salt, which are particularly but not exclusively suitable for the plasmids, are preferably those which correspond to the following formula:

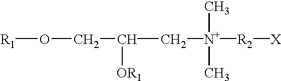

in which R₁ is a saturated or unsaturated linear aliphatic radical having 12 to 18 carbon atoms, R₂ is another aliphatic radical, containing 2 or 3 carbon atoms, and X is a hydroxyl or amine group.

Among these cationic lipids, preference is given to DMRIE (N- (2-hydroxyethyl)-N,N-dimethyl-2,3-bis(tetradecyloxy)-1-propanammonium; WO-A-96/34109), preferably associated with a neutral lipid, preferably DOPE (dioleoylphosphatidylethanolamine; Behr J. P., 1994, Bioconjugate Chemistry, 5, 382-389), to form DMRIE-DOPE.

The plasmid is preferably mixed with this adjuvant extemporaneously, and the mixture thus constituted is preferably given time to form complexes, for example for a period of time ranging from 10 to 60 minutes, in particular of the order of 30 minutes, before it is administered.

When DOPE is present, the DMRIE:DOPE molar ratio preferably ranges from 95:5 to 5:95, more particularly 1:1.

The plasmid:DMRIE or DMRIE-DOPE adjuvant weight ratio may in particular range from 50:1 to 1:10, in particular from 10:1 to 1:5, and preferably from 1:1 to 1:2.

The cytokine(s) (5) optionally present may be introduced into the composition or vaccine in the form of protein, or may be coexpressed in the host with the FIV protein(s). Preference is given to coexpression of the cytokine(s), either using the same vector as that expressing the proteins or using a separate vector.

These cytokines may in particular be selected from feline cytokines, especially those of cats, such as feline interleukin 18 (fIL-18) (Taylor S. et al., DNA Seq., 2000, 10(6), 387-394), fIL-16 (Leutenegger C. M. et al., DNA Seq., 1998, 9(1), 59-63), fIL-12 (Fehr D. et al., DNA Seq., 1997, 8(1-2), 77-82; Imamura T. et al., J. Vet. Med. Sci., 2000 62(10), 1079-1087) and feline GM-CSF (granulocyte macrophage colony-stimulating factor) (GenBank AF053007).

In accordance with the invention, the vaccination against FIV may be combined with vaccinations against other feline pathogenic agents. The other feline pathogenic agents are in particular feline rhinotracheitis virus or feline herpes virus (FHV), feline leukaemia viruses (FeLV type A and type B), feline parvoviruses (FPV), feline infectious peritonitis virus (FIPV), feline calicivirus (FCV), rabiesvirus, Chlamydia.

The preparations and vaccines according to the invention may in particular be administered parenterally, e.g. subcutaneously, intradermally and/or intramuscularly, or orally and/or nasally.

The various preparations and vaccines may be injected using a needleless liquid jet injector.

The immunogenic compositions and the vaccines according to the invention comprise an effective amount of plasmid or viral vector, the determination of these amounts being within the scope of those skilled in the art. The applicant recommends:

in the case of the plasmid-based immunogenic compositions or vaccines, a dose may comprise from approximately 1 μg to approximately 2 000 μg, in particular from approximately 50 μg to approximately 1 000 μg. The dose volumes may be between 0.1 and 2 ml, preferably between 0.2 and 1 ml;

in the case of the poxvirus-based immunogenic compositions or vaccines, a dose may be between approximately 10³ pfu and approximately 10⁹ pfu. When the vector is the canarypox virus, the dose is more particularly between approximately 10⁵ pfu and approximately 10⁹ pfu, preferably between approximately 10⁶ and approximately 10⁸ pfu. The dose volumes of the viral vector-based feline vaccines and immunogenic compositions are generally between 0.1 and 2.0 ml, preferably between 0.2 and 1.0 ml.

The kit may comprise the doses of vaccine to vaccinate either an animal or several animals.

According to a particular mode, the kit comprises two doses of plasmid-based vaccine per dose of viral vector-based vaccine.

The invention will now be described in greater detail using embodiments taken by way of nonlimiting examples.

### EXAMPLES

All the constructs are prepared using the standard molecular biology techniques (cloning, restriction enzyme digestion, synthesis of a single-stranded complementary DNA, polymerase chain reaction, oligonucleotide DNA polymerase-mediated elongation, etc.) described by Sambrook J. et al. (Molecular Cloning: A Laboratory Manual, 2nd Edition, Cold Spring Harbor Laboratory, Cold Spring Harbor, N.Y., 1989). All the restriction fragments used for the present invention, and also the various polymerase chain reaction (PCR) fragments, are isolated and purified using the “Geneclean®” kit (BIO101 Inc. La Jolla, Calif.).

The examples use the FIV Villefranche IFFA 1/88 strain (Steffan A. M. et al., J. Gen. Virol., 1994, 75, 3647-3653). It goes without saying that the invention may be applied to the other FIV strains. Mention may be made, for example, of the Petaluma strain (available from the American Type Culture Collection (ATCC) under the number VR-1312, and nucleotide sequence registered in GenBank under the number M25381; env gene: nucleotides 6266 to 8836; gag gene: nucleotides 628-1980; gag/pro: nucleotides 628-2336). One may also cite strain NCSU1 available from ATCC under reference VR2333. Reference may also be made to the articles by Sodora and by Bachmann (Sodora D. L. et al., J. Virol., 1994, 68(4), 2230-2238; Bachmann M. H. et al., J. Virol., 1997, 71(6), 4241-4253) which describe a certain number of FIV strains and indicate the references for access to the sequences in GenBank. Strain FIV-14 is referenced in Genbank under NC_{—}001482 (env gene: nucleotides 6266 to 8836; gag gene: nucleotides 628-1980; gag/pro: nucleotides 628-2336), strain BM3070 is referenced in Genbank under AF474246 (env gene: nucleotides 6272 to 8833; gag gene: nucleotides 634-1986), strain OMA is referenced in Genbank under U56928 (env gene: nucleotides 6506 to 9097; gag gene: nucleotides 679-2179), etc.

The one skilled in the art is able to determine the PCR probes useful to clone the genes from the FIV strain used. The PCR probes used in the following examples to clone env, gag/pro, rev and tat from Villefranche strain may be used on other strains such as Petaluma or be slightly adapted when appropriate.

### Example 1

### Culturing of the FIV Virus

In order for them to be amplified, feline immuno-deficiency viruses of the Villefranche IFFA 1/88 strain are cultured on Q201 cells (feline helper T lymphocytes; Willet B. et al., J. Gen. Virol., 1997, 78, 611-618).

The Q201 cells are cultured in 25-cm² Falcon flasks with MEM Eagle medium supplemented with 2 mM of glutamine, with 10% of calf serum, with 100 IU/ml of penicillin with 100 μg/ml of streptomycin and with 100 IU/ml of recombined human interleukin-2, containing approximately 100 000 cells per ml. The cells are cultured at +37° C.

After 3 days, the cell layer reaches confluence. The culture medium is then replaced and the FIV virus is added at 5 pfu/cell.

When the cytopathic effect (CPE) is complete (generally 48-72 hours after the start of culturing), the viral suspensions are harvested and then clarified by centrifugation and frozen at −80° C. 3 to 4 successive passages are generally required for the production of a viral batch. The viral batch is stored at −80° C.

### Example 2

### Extraction of the FIV Viral RNA

The viral RNA contained in 100 ml of viral suspension of the FIV Villefranche strain is extracted, after thawing, with the solutions of the “High Pure™ Viral RNA Kit” (Cat #1 858 882, Roche Molecular Biochemicals), according to the manufacturer's instructions for the extraction steps. The RNA pellet obtained at the end of the extraction is resuspended with 1 to 2 ml of RNase-free sterile distilled water.

### Example 3

### Construction of the Plasmid pPB371

The FIV complementary DNA (cDNA) is synthesized with the “Gene Amp RNA PCR Kit” (Cat # N 808 0017, Perkin-Elmer, Norwalk, Conn. 06859, USA) using the conditions given by the manufacturer.

A reverse transcription reaction, followed by a polymerase chain reaction (“RT-PCR” reaction) is carried out with 50 μl of the FIV viral RNA suspension (Example 2) and with the following oligonucleotideso:

| | |
|---|---|
| FC116 (36 mer) | (SEQ ID No.:1) |

5′ TTTTTTCTGCAGCAATAAGAATGGCAGAAGGATTTG 3′

| | |
|---|---|
| and FC117 (36 mer) | (SEQ ID No.:2) |

5′ TCGCACCTGAAACATCTCGAGTGTTTCCACATGTAT 3′.

This pair of oligonucleotides allows the incorporation of a PstI restriction site, of an XhoI restriction site and of an initiating ATG codon in 5′ of the insert.

The first cDNA strand is synthesized by elongation of the oligonucleotide FC117, after hybridization of the latter to the RNA matrix.

The conditions for synthesis of the first cDNA strand are a temperature of 42° C. for 15 min, then of 99° C. for 5 min and, finally, of 4° C. for 5 min. The conditions for the PCR reaction in the presence of the pair of oligonucleotides FC116 and FC117 are a temperature of 95° C. for 2 min, then 40 cycles (95° C. for 30 sec, then 50° C. for 45 sec, and 72° C. for 3 min) and, finally, 72° C. for 7 min, so as to produce a 1476 bp fragment.

This fragment is digested with the PstI restriction enzyme and then with the XhoI restriction enzyme so as to isolate, after agarose gel electrophoresis, the approximately 1450 pb PstI-XhoI fragment. This fragment is called fragment A.

A second reverse transcription reaction, followed by a polymerase chain reaction (“RT-PCR” reaction), is carried out with 50 μl of the FIV viral RNA suspension (Example 2) and with the following oligonucleotides:

| | |
|---|---|
| FC118 (36 mer) | (SEQ ID No.:3) |

5′ATACATGTGGAA4CACTCGAGATGTTTCAGGTGCGA 3′

| | |
|---|---|
| and FC119 (54 mer) | (SEQ ID No.:4) |

5′TTTTTTGGATCCCCCGGGCTGCAGGAATTCTGAGATACTTCATCATTC
C 3′.

This pair of oligonucleotides allows the incorporation of an XhoI restriction site, of a BamHI restriction site and of a stop codon in 3′ of the insert.

The first cDNA strand is synthesized by elongation of the oligonucleotide FC118, after hybridization of the latter to the RNA matrix.

The conditions for synthesis of the first cDNA strand are a temperature of 42° C. for 15 min, then of 99° C. for 5 min and, finally, of 4° C. for 5 min. The conditions for the PCR reaction in the presence of the pair of oligonucleotides FC118 and FC119 are a temperature of 95° C. for 2 min, then 40 cycles (95° C. for 130 sec, then 50° C. for 45 sec, and 72° C. for 3 min) and, finally, 72° C. for 7 min, so as to produce a 1193 bp fragment.

This fragment is digested with the XhoI restriction enzyme and then with the BamHI restriction enzyme so as to isolate, after agarose gel electrophoresis, the approximately 1170 bp XhoI-BamHI fragment. This fragment is called fragment B.

Fragments A and B are ligated with the eukaryotic expression plasmid pVR1012 (FIG. 1 and Example 7 of WO-A-98/03199; Hartikka J. et al., 1997, Human Gene Therapy, 7, 1205-1217) digested beforehand with XbaI and EcoRI, to give the plasmid pPB371 (7467 bp). This plasmid contains, under the control of the human cytomegalovirus immediate early, or hCMV-IE, promoter, an insert encoding the FIV env protein.

### Example 4

### Construction of the Plasmid pPB374

The FIV complementary DNA (cDNA) is synthesized with the “Gene Amp RNA PCR Kit” (Cat # N 808 0017, Perkin-Elmer, Norwalk, Conn. 06859, USA) using the conditions given by the manufacturer.

A reverse transcription reaction, followed by a polymerase chain reaction (“RT-PCR” reaction), is carried out with 50 μl of the FIV viral RNA suspension (Example 2) and with the following oligonucleotides:

| | |
|---|---|
| PB670 (37 mer) | (SEQ ID No.:5) |

5? TTTGTCGACMGGTAGGAGAGATTCTACAGCMCATG 3′

| | |
|---|---|
| and PE674 (40 mer) | (SEQ ID No.:6) |

5′ GCGGCCGCGTTATTGAGCCATTACTAACCTMTAG 3′.

This pair of oligonucleotides allows the incorporation of a SalI restriction site, of a NotI restriction site and of an initiating ATG codon in 5′ at the insert, and of a stop codon in 3′ at the insert.

The first cDNA strand is synthesized by elongation of the oligonucleotide PB674, after hybridization of the latter to the RNA matrix.

The conditions for synthesis of the first cDNA strand are a temperature of 42° C. for 15 min, then of 99° C. for 5 min and, finally, 4° C. for 5 min. The conditions for the PCR reaction in the presence of the pair of oligonucleotides PB670 and PB674 are a temperature of 95° C. for 2 min, then 40 cycles (95° C. for 30 sec, then 50° C. for 45 sec, and 72° C. for 3 min) and, finally, 72° C. for 7 min, so as to produce a 1758 bp fragment.

This fragment is digested with the SalI restriction enzyme and then with the NotI restriction enzyme so as to isolate, after agarose gel electrophoresis, the approximately 1750 bp SalI-NotI fragment. This fragment is ligated with the expression plasmid pVR1012 (Example 3) digested beforehand with SalI and NotI, to give the plasmid pPB374 (6633 bp). This plasmid contains, under the control of the hCMV-IE promoter, an insert encoding the FIV gag/pro proteins.

### Example 5

### Construction of the Plasmid pPB375

The FIV complementary DNA (cDNA) is synthesized with the “Gene Amp RNA PCR Kit” (Cat # N 808 0017, Perkin-Elmer, Norwalk, Conn. 06859, USA) using the conditions given by the manufacturer.

A reverse transcription reaction, followed by a polymerase chain reaction (“RT-PCR” reaction), is carried out with 50 μl of the FIV viral RNA suspension (Example 2) and with the following oligonucleotides:

FC116 (36 mer) (SEQ ID No.:1)

and FC120 (48 mer) (SEQ ID No.:7)

5′TTITTACCTGCATTTCCTTCTTCCAGTTTTACCTCTTGAATTTCGTTC 3′.

This pair of oligonucleotides allows the incorporation of a PstI restriction site, of a BspMI restriction site and of an initiating ATG codon in 5′ at the insert.

The first cDNA strand is synthesized by elongation of the oligonucleotide FC120, after hybridization of the latter to the RNA matrix.

The conditions for synthesis of the first cDNA strand are a temperature of 42° C. for 15 min, then of 99° C. for 5 min and, finally, of 4° C. for 5 min. The conditions for the PCR reaction in the presence of the pair of oligonucleotides FC116 and FC120 are a temperature of 95° C. for 2 min, then 40 cycles (95° C. for 30 sec, then 50° C. for 45 sec, and 72° C. for 3 min) and, finally, 72° C. for 7 min, so as to produce a 265 bp fragment.

This fragment is digested with the PstI restriction enzyme and then with the BspMI restriction enzyme so as to isolate, after agarose gel electrophoresis, the approximately 240 bp PstI-BspMI fragment. This fragment is called fragment C.

A second reverse transcription reaction, followed by a polymerase chain reaction (“RT-PCR” reaction), is carried out with 50 μl of the FIV viral RNA suspension (Example 2) and with the following oligonucleotides:

| | |
|---|---|
| PB672 (48 mer) | (SEQ ID No.:8) |

5′TTTACTGGAAGAAGGAAATGCAGGTAAAAGGAAAAGACAAAGAAGAAG
3′

| | |
|---|---|
| and PB673 (36 mer) | (SEQ ID No.:9) |

5′TTTAGATCTTTAGTCCATAAGCATTCTTTCTATTTC 3′.

This pair of oligonucleotides allows the incorporation of a BspMI restriction site, of a BglII restriction site and of a stop codon in 3′ of the insert.

The first cDNA strand is synthesized by elongation of the oligonucleotide PB673, after hybridization of the latter to the RNA matrix.

The conditions for synthesis of the first cDNA strand are a temperature of 42° C. for 15 min, then of 99° C. for 5 min and, finally, of 4° C. for 5 min. The conditions for the PCR reaction in the presence of the pair of oligonucleotides PB672 and PB673 are a temperature of 95° C. for 2 min, then 40 cycles (95° C. for 30 sec, then 50° C. for 45 sec, and 72° C. for 3 min) and, finally, 72° C. for 7 min, so as to produce a 246 bp fragment.

This fragment is digested with the BspMI restriction enzyme and then with the BglII restriction enzyme so as to isolate, after agarose gel electrophoresis, the approximately 230 bp BspMI-BglII fragment. This fragment is called fragment D.

Fragments C and D are ligated with the expression plasmid pVR1012 (Example 3) digested beforehand with the PstI and BglII restriction enzymes, to give the plasmid pPB375 (5316 bp). This plasmid contains, under the control of the hCMV-IE promoter, an insert encoding the FIV rev protein.

### Example 6

### Construction of the Plasmid pPB383

The FIV complementary DNA (cDNA) is synthesized with the “Gene Amp RNA PCR Kit” (Cat # N 808 0017, Perkin-Elmer, Norwalk, Conn. 06859, USA) using the conditions given by the manufacturer.

A reverse transcription reaction, followed by a polymerase chain reaction (“RT-PCR” reaction), is carried out with 50 μl of the FIV viral RNA suspension (Example 2) and with the following oligonucleotides:

| | |
|---|---|
| PB680 (29 mer) | (SEQ ID No.:10) |

5′TTTCTGCAGATGGAAGACATAATAGTATT 3′,

| | |
|---|---|
| and PB681 (32 mer) | (SEQ ID No.:11) |

5′TTTAGATCTCTAAGCAGTAGTTATTGATAATG 3′.

This pair of oligonucleotides allows the incorporation of a BglII restriction site, of a PstI restriction site and of an initiating ATG codon in 5′ of the insert, and of a stop codon in 3′ of the insert.

The first cDNA strand is synthesized by elongation of the oligonucleotide PB681, after hybridization of the latter to the RNA matrix.

The conditions for synthesis of the first cDNA strand are a temperature of 42° C. for 15 min, then of 99° C. for 5 min and, finally, of 4° C. for 5 min. The conditions for the PCR reaction in the presence of the pair of oligonucleotides PB680 and PB681 are a temperature of 95° C. for 2 min, then 40 cycles (95° C. for 30 sec, then 50° C. for 45 sec, and 72° C. for 1 min) and, finally, 72° C. for 7 min, so as to produce a 254 bp fragment.

This fragment is digested with the PstI restriction enzyme and then with the BglII restriction enzyme so as to isolate, after agarose gel electrophoresis, the approximately 240 bp PstI-BglII fragment. This fragment (fragment E) is ligated with the expression plasmid pVR1012 (Example 3) digested beforehand with PstI and BglII, to give the plasmid pPB383 (5089 bp). This plasmid contains, under the control of the hCMV-IE promoter, an insert encoding the FIV tat protein.

### Example 7

### Construction of the Recombined Viruses vCP242, vCP253 and vCP255

Patent WO-A-98/21354 describes, in detail, the production of the recombined viruses vCP242, vCP253 and vCP255, respectively in Examples 1, 2 and 4.

The recombined virus vCP242 comprises the nucleotide sequence encoding the env protein of the FIV Villefranche strain, under the control of an H6 promoter of the vaccinia virus and inserted into the ALVAC canarypox virus C6 site.

The recombined virus vCP253 comprises the nucleotide sequence encoding the gag/pro proteins of the FIV Villefranche strain, under the control of an 13L promoter of the vaccinia virus and inserted into the ALVAC canarypox virus C6 site.

The recombined virus vCP255 comprises the nucleotide sequence encoding the env protein of the FIV Villefranche strain, under the control of an H6 promoter of the vaccinia virus, and the nucleotide sequence encoding the gag/pro proteins of the FIV Villefranche strain, under the control of an 13L promoter of the vaccinia virus, both inserted into the ALVAC canarypox virus C6 site.

### Example 8

### Construction of the Donor Plasmid for the Insertion into the ALVAC Canarypox Virus C5 Site

FIG. 16 of patent U.S. Pat. No. 5,756,103 shows the sequence of a 3199 bp fragment of the genomic DNA of the canarypox virus. Analysis of this sequence revealed an open reading frame (ORF), which was called C5L, which begins at position 1538 and ends at position 1859. The construction of a plasmid with an insertion resulting in the deletion of ORF C5L, and the replacement thereof with a multiple cloning site flanked by transcription and translation stop signals, was carried out as described below.

A PCR reaction was carried out on the matrix consisting of the genomic DNA of the canarypox virus, and with the following oligonucleotides:

| | |
|---|---|
| C5A1 (42 mer) | (SEQ ID No.:12) |

5′ATCATCGAGCTCCAGCTGTAATTCATGGTCGAAAAGAAGTGC 3′

| | |
|---|---|
| and FC121 (79 mer): | (SEQ ID No.:13) |

5′GAATTCCTCGAGAGATCTCTGCAGCCCGGGTTTTTATAGCTAATTAGT
CATTTTTTGAGAGTACCACTTCAGCTACCTC 3′

so as to isolate a 229 bp PCR fragment (fragment B).

A PCR reaction was carried out on the matrix consisting of the genomic DNA of the canarypox virus, and with the following oligonucleotides:

| | |
|---|---|
| FC122 (78 mer): | (SEQ ID No.:14) |

5′CCCGGGCTGCAGAGATCTCTCGAGGAATTCTTTTTATTGATTAACTAG
TCATTATAAAGATCTAAAATGCATAATTTC 3′

| | |
|---|---|
| and C5D1 (45 mer) | (SEQ ID No.:15) |

5′GATGATGGTACCGTAAACAAATATAATGAAAGTATTCTAAACTA 3′

so as to isolate a 488 bp PCR fragment (fragment C).

Fragments B and C were hybridized together so as to serve as a matrix for a PCR reaction carried out with the oligonucleotides C5A1 (SEQ ID No.:12) and C5D1 (SEQ ID No.:15), to generate a 693 bp PCR fragment. This fragment was digested with the SacI and KpnI restriction enzymes so as to isolate, after agarose gel electrophoresis, a 676 bp SacI-KpnI fragment. This fragment was ligated with the vector pBlueScript® II SK+ (Stratagene, La Jolla, Calif., USA, Cat # 212205), digested beforehand with the SacI and KpnI restriction enzymes, to give the plasmid pFC115. The sequence of this plasmid was verified by sequencing. This plasmid contains 166 bp of sequences located upstream of ORF C5L (“C5 left flanking arm”), a vaccinia early transcription stop signal, stop codons in the 6 reading frames, a multiple cloning site containing the SmaI, PstI, BglII, XhoI and EcoRI restriction sites and, finally, 425 bp of sequences located downstream of ORF C5L (“C5 right flanking arm”).

The plasmid pMP528HRH (Perkus M. et al. J. Virol. 1989, 63, 3829-3836) was used as matrix to amplify the complete sequence of the vaccinia H6 promoter (GenBank accession No. M28351) with the following oligonucleotides:

| | |
|---|---|
| JCA291 (34 mer) | (SEQ ID No.:16) |

5′AAACCCGGGTTCTTTATTCTATACTTAAAAAAGTG 3′

| | |
|---|---|
| and JCA292 (43 mer) | (SEQ ID No.:17) |

5′AAAAGAATTCGTCGACTACGATACAAACTTAACGGATATCGCG 3′

so as to amplify a 149 bp PCR fragment. This fragment was digested with the SmaI and EcoRI restriction enzymes so as to isolate, after agarose gel electrophoresis, a 138 bp SmaI-EcoRI restriction fragment. This fragment was then ligated with the plasmid pFC115, digested beforehand with SmaI and EcoRI, to give the plasmid pFC116.

### Example 9

### Construction of the Donor Plasmid for the Insertion into the ALVAC Canarypox Virus C6 Site

FIG. 4 of patent WO-A-01/05934 shows the sequence of a 3700 bp fragment of the genomic DNA of the canarypox virus. Analysis of this sequence revealed an open reading frame (ORF), which was called C6L, which begins at position 377 and ends at position 2254. The construction of a plasmid with an insertion resulting in the deletion of ORF C6L, and in the replacement thereof with a multiple cloning site flanked by transcription and translation stop signals, was carried out as described below.

A PCR reaction was carried out on the matrix consisting of the genomic DNA of the canarypox virus, and with the following oligonucleotides:

| | |
|---|---|
| CSA1 (42 mer): | (SEQ ID No.:18) |

5′ATCATCGAGCTCGCGGCCGCCTATCAAAAGTCTTAATGAGTT 3′

| | |
|---|---|
| and FC123 (79 mer): | (SEQ ID No.:19) |

5′GAATTCCTCGAGAGATCTCTGCAGCCCGGGTTTTTATAGCTAATTAGT
CATTTTTCGTAAGTAAGTATTTTTATTTAA 3′

so as to isolate a 438 bp PCR fragment (fragment D).

A PCR reaction was carried out on the matrix consisting of the genomic DNA of the canarypox virus, and the following oligonucleotides:

| | |
|---|---|
| FC124 (78 mer) | (SEQ ID No.:20) |

5′CCCGGGCTGCAGAGTCTCTCGAGGAATTCTTTTTTATTGATTAACTAG
TCAAATGAGTATATATAATTGAAAAAAGTAA 3′

| | |
|---|---|
| and C6D1 (45 mer) | (SEQ ID No.:21) |

5′GATGATGGTACCTTCATAAATACAAGTTTGATTAAACTTAAGTTG 3′

so as to isolate a 1216 bp PCR fragment (fragment E).

Fragments D and E were hybridized together so as to serve as a matrix for a PCR reaction carried out with the oligonucleotides C6A1 (SEQ ID No.:18) and C6D1 (SEQ ID No. :21), so as to generate a 1642 bp PCR fragment. This fragment was digested with the SacI and KpnI restriction enzymes so as to isolate, after agarose gel electrophoresis, the 1625 bp SacI-KpnI fragment. This fragment was ligated with the vector pBlueScript® II SK+ (Stratagene, La Jolla, Calif., USA, Cat # 212205), digested beforehand with the SacI and KpnI restriction enzymes, to give the plasmid pFC117. The sequence of this plasmid was verified by sequencing. This plasmid contains 370 bp of sequences located upstream of ORF C6L (“C6 left flanking arm”), a vaccinia early transcription stop signal, stop codons in the 6 reading frames, a multiple cloning site containing the SmaI, PstI, BglII, XhoI and EcoRI restriction sites and, finally, 1156 bp of sequences located downstream of ORF C6L (“C6 right flanking arm”).

The plasmid pMPIVC (Schmitt J. F. C. et al, J. Virol., 1988, 62, 1889-1897; Saiki R. K. et al., Science, 1988, 239, 487-491) was used as matrix to amplify the complete sequence of the vaccinia I3L promoter with the following oligonucleotides:

| | |
|---|---|
| FC112 (33 mer) | (SEQ ID No.:22) |

5′AAACCCGGGCGGTGGTTTGCGATTCCGAAATCT 3′ and

| | |
|---|---|
| FC113 (43 mer) | (SEQ ID No.:23) |

5′AAAAGAATTCGGATCCGATTAAACCTAAATAATTGTACTTTGT 3′

so as to amplify a 151 bp PCR fragment. This fragment was digested with the SmaI and EcoRI restriction enzymes so as to isolate, after agarose gel electrophoresis, an approximately 136 bp SmaI-EcoRI restriction fragment. This fragment was then ligated with the plasmid pFC117, digested beforehand with SmaI and EcoRI, to give the plasmid pFC118.

### Example 10

### Construction of the Recombined Virus vCP1719

Fragments C and D (Example 5) were ligated with the plasmid pFC116 (Example 8), digested beforehand with the PstI and BglII restriction enzymes, to give the plasmid pFC119.

Fragment E (Example 6) was ligated with the plasmid pFC118 (Example 9), digested beforehand with the PstI and BglII restriction enzymes, to give the plasmid pFC120.

The plasmid pFC120 was linearized with NotI, and then transfected into primary chick embryo cells infected with canarypox virus (ALVAC strain) according to the previously described calcium phosphate precipitation technique (Panicali and Paoletti Proc. Nat. Acad. Sci. 1982, 79, 4927-4931; Piccini et al. In Methods in Enzymology, 1987, 153, 545-563. Eds. Wu R. and Grossman L. Academic Press). Positive plaques were selected on the basis of hybridization with a radiolabelled probe specific for the nucleotide sequence of the tat protein. These plaques underwent 4 successive plaque selection/purification cycles until a pure population had been isolated. A plaque representative of in vitro recombination between the donor plasmid pFC120 and the genome of the ALVAC canarypox virus was then amplified and the stock of recombined virus obtained was named vCP1719.

Optionally, the recombined viruses obtained were used for a second transfection into primary chick embryo cells in the presence of the plasmid pFC119 linearized with NotI, according to the calcium phosphate precipitation technique. Positive plaques were selected on the basis of hybridization with a radiolabelled probe specific for the nucleotide sequence of the rev protein. These plaques underwent 4 successive plaque selection/purification cycles until a pure population had been isolated. A plaque representative of in vitro recombination between the donor plasmids pFC119 and pFC120 and the genome of the ALVAC canarypox virus was then amplified and the stock of recombined virus obtained was named vCP1720.

### Example 11

### Construction of the Plasmid pJP090

Cat blood was harvested in a tube containing EDTA, via a blood sample taken from the jugular vein. The mononuclear cells were harvested by centrifugation on a Ficoll gradient, and then cultured in Petri dishes 60 mm in diameter. The cat mononuclear cells in culture were then stimulated either with concanavalin A (conA) (final concentration of approximately 5 μg/ml) or with phytohaemagglutinin (PHA) (final concentration of approximately 10 μg/ml). After stimulation, the “ConA” and “PHA” lymphoblasts were harvested by scraping the culture dishes, and the total RNA from these cells was extracted using the “mRNA isolation kit for white blood cells” (Boehringer Mannheim/Roche Cat # 1 934 325).

The total RNA extracted from the cat lymphocytes stimulated with the ConA or with PHA was used as a matrix for synthesizing the first complementary DNA strand. This first complementary DNA strand was produced by elongation of the oligonucleotide p(dT)15 (Boehringer Mannheim/Roche Cat # 814 270). The single-stranded complementary DNA obtained was then used as a matrix for a PCR reaction with the following oligonucleotides:

| | |
|---|---|
| FC125 (48 mer) | (SEQ ID No.:24) |

5′TTTTTTGCGGCCGCCACCATGTGGCTGCAGAACCTGCTTTTCCTG
GGC 3′ and

| | |
|---|---|
| FC126 (50 mer) | (SEQ ID No.:25): |

5′TTTTTTGCGGCCGCTACGTATCACTTCTTGACTGGTTTCCAGCAGTC
AAA 3′

so as to amplify an approximately 473 base pair (bp) PCR fragment. This fragment was purified by agarose gel electrophoresis. This fragment was then digested with NotI and the approximately 453 bp NotI-NotI fragment thus obtained was ligated with the plasmid pVR1012 (Example 3), digested beforehand with NotI, to give the plasmid pJPO90 (5365 bp). The direction of the insert in pJPO90 was verified. The NotI-NotI fragment cloned on this plasmid was completely sequenced. This sequence (SEQ ID No. 26), which encodes a 144 amino acid protein (SEQ ID No. 27), is the feline GM-CSF cytokine.

### Example 12

### Production of DNA Vaccines

A solution of DNA containing the plasmid pPB371 (Example 3) is concentrated by ethanol precipitation as described in Sambrook et al. (1989). The DNA residue is taken up with a 1.8% NaCl solution so as to obtain a concentration of 1 mg/ml. A 0.75 mM DMRIE-DOPE solution is prepared by taking up a DMRIE-DOPE lyophilisate with a suitable volume of sterile H₂O.

The plasmid DNA-lipid complexes are formed by diluting, in equal amounts, the 0.75 mM solution of DMRIE-DOPE (1:1) with the 1 mg/ml solution of DNA in 1.8% NaCl. The DNA solution is introduced gradually, using a 26G crimped needle, along the wall of the flask containing the cationic lipid solution so as to avoid the formation of foam. As soon as the two solutions are mixed, gentle stirring is carried out. At the end of this procedure, a composition is obtained which comprises 0.375 mM of DMRIE-DOPE and 500 μg/ml of plasmid.

It is desirable for all the solutions used to be at ambient temperature for all of the operations described above. The DNA/DMRIE-DOPE complexation is left to develop at ambient temperature for 30 minutes, before immunizing the animals.

DNA vaccines may also be produced with solutions of DNA containing the plasmids pPB374 (Example 4), pPB375 (Example 5), pPB383 (Example 6), pJP090 (Example 11) or mixtures of at least two of these 5 plasmids, according to the technique described in the present example.

### Example 13

### In Vitro Expression Tests

Expression of the FIV proteins is tested for each construct, using the conventional methods of indirect immunofluorescence and Western blotting.

These tests are carried out on Petri dishes containing CHO cells cultured in monolayers and transfected with plasmids, or containing CEF cells cultured in monolayers and infected with recombined viruses.

The FIV proteins are detected using labelled antisera and sera from infected cats.

The size of the fragments obtained after migration on agarose gel is compared to those expected.

### Example 14

### Effectiveness on Animals

SPF Hillgrove cats (Biological Laboratories Europe Ltd.) without anti-FIV antibodies, approximately 12 weeks old, are randomly divided into three groups of 6 animals.

The cats of the first group (group A) are vaccinated on D0 and D28 by intraamuscular administration of 1 ml of a mixture of plasmids pPB371 (Example 3) and pPB374 (Example 4), and then administration of a booster on D56 by intramuscular injection of 1 ml of recombined virus vCP255 (Example 7) at a titre of 10^{8.0} TCID₅₀/ml.

The cats of the second group (group B) are vaccinated on D0 and D28 by intramuscular administration of 1 ml of a mixture of the plasmids pPB371 and pPB374, formulated with DMRIE-DOPE (Example 12), and then administration of a booster on D56 by intramuscular injection of 1 ml of recombined virus vCP255 (Example 7) at a titre of 10^{8.0} TCID₅₀/ml.

The concentration of total DNA in the DNA vaccines is 200 μg/ml, i.e. 100 μg/ml for each plasmid contained in the mixture.

The lipid/DNA molar ratio for the DNA vaccines formulated with DMRIE-DOPE is 0.25.

The third group (controls) is the control group (no vaccination, challenge on D84).

All the cats are challenged on D84 by intraperitoneal administration of 1 ml of pathogenic FIV virus (Petaluma strain) at a titre of 25 CID₅₀/ml (CID being 50% infectious dose in cats).

Firstly, the viraemia assessed by viral re-isolation and PCR was observed, as was the antibody response.

Viral re-isolation from week 4 after challenge to week 16 (number of animals exhibiting positive viraemia):

| Groups | Viral re-isolation | PCR |
|---|---|---|
| Group A | 2/6 | 2/6 |
| Group B | 3/6 | 2/6 |
| Controls | 6/6 | 5/6 |

The viral re-isolation is carried out by coculturing approximately 5×10⁶ peripheral blood mononuclear cells (PBMCs) with approximately 10⁶ MYA-1 cells in RPMI 1640 medium for 21 days. The presence of FIV proviral DNA present in the PBMC cells is identified by PCR.

A lack of viraemia is observed in 67% of the animals of group A and 50% of group B.

Cellular response on the day of the challenge and 4 weeks after challenge (number of animals exhibiting a positive CTL response):

| Antigen | Gag | Gag | Env | Env |
|---|---|---|---|---|
| detected | Week 0 | Week 4 | Week 0 | Week 4 |
| Group A | 1/6 | 4/6 | 0/6 | 2/6 |
| Group B | 2/6 | 6/6 | 2/6 | 0/6 |
| Controls | 0/5* | 2/6 | 0/5* | 0/6 |

*It was not possible to take samples from one animal.

Skin fibroblasts are taken, by biopsy, from each of the cats. The fibroblasts are labelled with ⁵¹Cr and then infected with a vaccinia recombinant expressing either env or gag, in the presence of PBMCs originating from each of the cats. The cytotoxic (CTL) response is measured by ⁵¹Cr release.

It is observed that the vaccination stimulates (priming effect) the cytotoxic response for the groups of vaccinated animals, particularly for gag.

Humoral response after challenge (number of animals having a positive anti-TM serological response by ELISA):

| Week | 0 | 2 | 4 | 8 | 12 | 16 |
|---|---|---|---|---|---|---|
| Group A | 0/6 | 3/6 | 4/6 | 3/6 | 3/6 | 5/6 |
| Group B | 0/6 | 3/6 | 5/6 | 5/6 | 4/6 | 6/6 |
| Controls | 0/6 | 0/6 | 0/6 | 3/6 | 5/6 | 5/6 |

This is an ELISA to quantify the antibodies using a peptide corresponding to the major epitope of the transmembrane (TM) protein.

The vaccination stimulates (priming effect) the humoral immune response.

It should be clearly understood that the invention defined by the attached claims is not limited to the particular embodiments indicated in the above description, but encompasses the variants which depart neither from the context nor from the spirit of the present invention.

1. A kit for immunizing Felidae against FIV, comprising, packaged separately:
a first immunogenic composition comprising, in a pharmaceutically acceptable vehicle or excipient, a plasmid containing and expressing, in vivo, a polynucleotide encoding an FIV protein chosen among the group consisting of env, gag and gag/pro,
a second immunogenic composition comprising, in a pharmaceutically acceptable vehicle or excipient, a viral vector containing and expressing in vivo, a polynucleotide encoding an FIV protein chosen among the group consisting of env, gag and gag/pro, with the condition according to which a same FIV protein chosen among the group consisting of env, gag and gag/pro, is encoded by both the plasmid and the viral vector.

2. The kit according to claim 1, wherein the plasmid and the viral vector comprise the polynucleotides encoding env and gag/pro.

3. The kit according to claim 1, wherein the plasmid and the viral vector comprise the polynucleotides encoding env and gag.

4. The kit according to claim 1, wherein the viral vector is an avipox virus.

5. The kit according to claim 1, wherein the viral vector is a canarypox virus.

6. The kit according to claim 1, wherein the viral vector is a fowlpox virus.

7. The kit according to claim 1, wherein the viral vector is an attenuated mutant of the vaccinia virus.

8. A kit for immunising Felidae against FIV, comprising, packaged separately:
a first immunogenic composition comprising, in a pharmaceutically acceptable vehicle or excipient, a plasmid containing and expressing, in vivo, a polynucleotide encoding FIV proteins env and gag/pro,
a second immunogenic composition comprising, in a pharmaceutically acceptable vehicle or excipient, a viral vector containing and expressing in vivo, a polynucleotide encoding FIV proteins env and gag/pro,
with the condition according to which a same FIV protein chosen among the group consisting of env and gag/pro, is encoded by both the plasmid and the viral vector.

9. The kit according to claim 8, wherein the viral vector is an avipox virus.

10. The kit according to claim 8, wherein the viral vector is a canarypox virus.

11. The kit according to claim 8, wherein the viral vector is a fowlpox virus.

12. The kit according to claim 8, wherein the viral vector is an attenuated mutant of the vaccinia virus.

13. A kit for immunising Felidae against FIV, comprising, packaged separately:
a first immunogenic composition comprising, in a pharmaceutically acceptable vehicle or excipient, a plasmid containing and expressing, in vivo, a polynucleotide encoding FIV proteins env and gag,
a second immunogenic composition comprising, in a pharmaceutically acceptable vehicle or excipient, a viral vector containing and expressing in vivo, a polynucleotide encoding FIV proteins env and gag,
with the condition according to which a same FIV protein chosen among the group consisting of env and gag, is encoded by both the plasmid and the viral vector.

14. The kit according to claim 13, wherein the viral vector is an avipox virus.

15. The kit according to claim 13, wherein the viral vector is a canarypox virus.

16. The kit according to claim 13, wherein the viral vector is a fowlpox virus.

17. The kit according to claim 13, wherein the viral vector is an attenuated mutant of the vaccinia virus.

18. The kit according to claim 1, wherein the first and/or second immunogenic composition comprise an adjuvant.

19. The kit according to claim 8, wherein the first and/or second immunogenic composition comprise an adjuvant.

20. The kit according to claim 13, wherein the first and/or second immunogenic composition comprise an adjuvant.

21. A method for immunizing a Felidae against FIV, comprising:
first administering to the Felidae a first immunogenic composition comprising, in a pharmaceutically acceptable vehicle or excipient, a plasmid containing and expressing, in vivo, a polynucleotide encoding an FIV protein chosen among the group consisting of env, gag and gag/pro,
then administering to the same Felidae a second immunogenic composition comprising, in a pharmaceutically acceptable vehicle or excipient, a viral vector containing and expressing in vivo, a polynucleotide encoding an FIV protein chosen among the group consisting of env, gag and gag/pro,
with the condition according to which a same FIV protein chosen among the group consisting of env, gag and gag/pro, is encoded by both the plasmid and the viral vector.

22. The method of claim 21, wherein the plasmid and the viral vector comprise the polynucleotides encoding env and gag/pro.

23. The method of claim 21, wherein the plasmid and the viral vector comprise the polynucleotides encoding env and gag.

24. The method of claim 21, wherein the viral vector is an avipox virus.

25. The method of claim 21, wherein the viral vector is a canarypox virus.

26. The method of claim 21, wherein the viral vector is a fowlpox virus.

27. The method of claim 21, wherein the viral vector is an attenuated mutant of the vaccinia virus.

28. The method of claim 21, wherein the second immunogenic composition is administered from 3 to 6 weeks after the administration of the first immunogenic composition.

29. The method of claim 21, wherein the second immunogenic composition is administered 4 weeks after the administration of the first immunogenic composition.

30. The method of claim 21, wherein the first immunogenic composition is administered twice before the second immunogenic composition is administered.

31. The method of claim 30, wherein the first immunogenic compositions are administered with a delay of 3 to 6 weeks between them.

32. The method of claim 21, wherein one administers to the same Felidae an annual boost with an immunogenic composition comprising, in a pharmaceutically acceptable vehicle or excipient, a viral vector containing and expressing in vivo, a polynucleotide encoding an FIV protein chosen among the group consisting of env, gag and gag/pro.

33. A method for immunizing a Felidae against FIV, comprising:
first administering to the Felidae a first immunogenic composition comprising, in a pharmaceutically acceptable vehicle or excipient, a plasmid containing and expressing, in vivo, a polynucleotide encoding FIV proteins env and gag/pro,
then administering to the same Felidae a second immunogenic composition comprising, in a pharmaceutically acceptable vehicle or excipient, a viral vector containing and expressing in vivo, a polynucleotide encoding FIV proteins env and gag/pro,
with the condition according to which a same FIV protein chosen among the group consisting of env and gag/pro, is encoded by both the plasmid and the viral vector.

34. The method of claim 33, wherein the viral vector is an avipox virus.

35. The method of claim 33, wherein the viral vector is a canarypox virus.

36. The method of claim 33, wherein the viral vector is a fowlpox virus.

37. The method of claim 33, wherein the viral vector is an attenuated mutant of the vaccinia virus.

38. The method of claim 33, wherein the second immunogenic composition is administered from 3 to 6 weeks after the administration of the first immunogenic composition.

39. A method for immunizing a Felidae against FIV, comprising:
first administering to the Felidae a first immunogenic composition comprising, in a pharmaceutically acceptable vehicle or excipient, a plasmid containing and expressing, in vivo, a polynucleotide encoding FIV proteins env and gag,
then administering to the same Felidae a second immunogenic composition comprising, in a pharmaceutically acceptable vehicle or excipient, a viral vector containing and expressing in vivo, a polynucleotide encoding FIV proteins env and gag,
with the condition according to which a same FIV protein chosen among the group consisting of env and gag, is encoded by both the plasmid and the viral vector.

40. The method of claim 39, wherein the viral vector is an avipox virus.

41. The method of claim 39, wherein the viral vector is a canarypox virus.

42. The method of claim 39, wherein the viral vector is a fowlpox virus.

43. The method of claim 39, wherein the viral vector is an attenuated mutant of the vaccinia virus.

44. The method of claim 39, wherein the second immunogenic composition is administered from 3 to 6 weeks after the administration of the first immunogenic composition.

45. The kit according to claim 21, wherein the first and/or second immunogenic composition comprise an adjuvant.

46. The kit according to claim 33, wherein the first and/or second immunogenic composition comprise an adjuvant.

47. The kit according to claim 39, wherein the first and/or second immunogenic composition comprise an adjuvant.
